(19) United States
(12) Patent Application Publication
(10) Pub. No.: US 2022/0328170 A1
(43) Pub. Date: Oct. 13, 2022

(54) PROVISION OF MEDICAL INSTRUMENTS

(71) Applicant: CARETAG APS, Hørsholm (DK)

(72) Inventor: Søren BILSØE, Espergaerde (DK)

(21) Appl. No.: 17/637,271

(22) PCT Filed: Aug. 21, 2020
(86) PCT No.: PCT/EP2020/073534

§ 371 Date: Feb. 22, 2022

(30) Foreign Application Priority Data
Aug. 23, 2019 (EP) 19193443.9

(51) Int. Cl. G16H 40/20; A61B 34/30; A61B 50/33; A61B 90/98; G06K 7/10; B65G 47/90

(52) U.S. Cl. CPC G16H 40/20; A61B 34/30; A61B 50/33; A61B 90/98; G06K 7/10366; B65G 47/90

(57) ABSTRACT

A method for providing one or more sets of medical instruments is provided, comprising: receiving a plurality of non-sorted medical instruments at an initial location, wherein each of the non-sorted medical instruments comprises an electronic identification tag, e.g. an RFID tag; identifying with an electronic reader a first medical instrument of a first type from the plurality of non-sorted medical instruments; and in accordance with one or more primary medical instruments including one or more medical instruments of the first type: repositioning the first medical instrument to a primary location for forming the primary set of medical instruments; and releasing the first medical instrument at the primary location.

The present disclosure relates to provision, such as packing and/or sorting, of medical instruments, e.g. surgical instruments, particularly to a system and method for providing medical instruments. For example, in preparation for a medical procedure.

### BACKGROUND

In an operating room, both certain surgical instruments and a certain number of instruments may be needed to perform a surgical procedure. Typically the instruments brought into the operating room prior to an operation is counted and registered manually, to be certain that the right amount and correct instruments are available for operation. Typically the instruments needed for a certain procedure is provided in a tray wherein the instruments are ordered for the specific procedure.

Manually sorting and packing equipment, before and after a procedure, is both time consuming and ineffective. Further, the consequence of errors may in a worst-case scenario pose a life-threatening situation for the patient.

Thus, it is advantageous if the handling, such as sorting and/or packing, equipment such as medical instruments can be automated. However, several problems exist in realising a system able to reliably and automatically identify and pack medical instruments in trays to reliably provide trays containing the right type and the right quantity of instruments to be used in different medical procedures.

Radio-frequency identification (RFID) may comprise passive chips, or “tags” as it is more commonly known, which essentially are small radio transceivers powered by the electromagnetic field emitted by an antenna. The received energy is used to transmit the content of a memory. Hence, the tag itself does not need to comprise a power source such as a battery.

RFID tags, such as passive RFID tags, may be in sizes of millimetres and may be reliably read from a distance of a couple of meters. However, these sizes and ranges are continuously improved. The reading range also depends on a number of factors such as surrounding material, orientation of the tag, transmission power of the antenna, number of tags etc. In the following, RFID tags will mainly be thought of as passive RFID tags. However, most apparatuses may be realized in ways of utilizing passive as well as active RFID tags.

By providing available instruments with an RFID tag, it is in theory possible to electronically read and identify a certain instrument. However, problems exist in realising a system that is able to reliably identify, retrieve and reposition an instrument comprising a specific RFID tag among a plurality of instruments comprising a plurality of RFID tags. Particularly, if the plurality of instruments is not organised. For example, after sterilization of instruments it is not uncommon that a plurality of random instruments is chaotically arranged in a tray.

Thus, there is a need for inventions that will provide high precision and reliability of automatically or semi-automatically provision of medical instruments.

### SUMMARY

An object of the present disclosure is to provide a method and a system that overcomes at least some problems of the present state of the art with respect to provision of medical instruments, such as, but not limited to, the abovementioned problems.

A further object of the present disclosure is to provide a method and a system that facilitates a system of high precision and reliability for automatic or semi-automatic provision of medical instruments. Thus, the present disclosure relates to method and a system for facilitating automation of handling of medical instruments.

Accordingly, a method for providing one or more sets of medical instruments is disclosed. The one or more sets of medical instruments includes a primary set of medical instruments and optionally a secondary set of medical instruments and/or a tertiary set of medical instruments.

Also a system is disclosed, such as a system for performing the disclosed method, such as a system for providing one or more sets of medical instruments including a primary set of medical instruments. The system comprises a first reposition mechanism, e.g. a gripper or suction device, configured to reposition a medical instrument comprising an electronic identification tag, e.g. an RFID tag. The system further comprises an electronic reader configured to read electronic identification tags at an identifier location by emitting and receiving electromagnetic radiation within a first frequency range. The first frequency range may be in the range of 860-960 MHz.

The method comprises receiving one or more requests indicative of one or more, such as a plurality of, medical instruments of the one or more sets of medical instruments. For example, the requests may be based on scheduled procedures at a hospital, provision for storage according to an inventory list, or similar. The one or more requests includes a primary request indicative of one or more, such as a plurality of, primary medical instruments to be included in the primary set of medical instruments. The one or more requests may include a secondary request indicative of one or more, such as a plurality of, secondary medical instruments to be included in the secondary set of medical instruments. The one or more requests may include a tertiary request indicative of one or more, such as a plurality of, tertiary medical instruments to be included in the tertiary set of medical instruments. The method may comprise receiving a plurality of requests. The method may comprise handling of a plurality of requests simultaneously. The primary medical instruments, the secondary medical instruments, and/or the tertiary medical instruments may include at least two different types of instruments, such as at least three different types of instruments, such as at least four different types of instruments.

The method comprises receiving a plurality of non-sorted medical instruments at an initial location. For example, a tray or a plurality of trays of random instruments may be received following sterilisation. Each of the non-sorted medical instruments comprises an electronic identification tag, e.g. an RFID tag. The plurality of non-sorted medical instruments may be received after being washed and/or prior to being sterilised, e.g. in an autoclave.

The method comprises identifying a first medical instrument of a first type from the plurality of non-sorted medical instruments. The identification may be provided by an electronic reader, such as the electronic reader of the disclosed system, configured for reading electronic identification tags by emitting and receiving electromagnetic radiation within the first frequency range.

The method comprises, in accordance with the primary medical instruments including one or more medical instruments of the first type, repositioning the first medical instrument to a primary location for forming the primary set of medical instruments; and releasing the first medical instrument at the primary location. For example, the method may comprise determining whether the primary medical instruments include one or more medical instruments of the first type, e.g. to decide whether to reposition and release the first medical instrument to the primary location. The primary location may correspond to a primary tray.

The method may comprise, in accordance with the primary medical instruments not including one or more medical instrument of the first type and the secondary medical instruments including one or more medical instruments of the first type, repositioning the first medical instrument to a secondary location for forming the secondary set of medical instruments; and releasing the first medical instrument at the secondary location. For example, the method may comprise determining whether the primary medical instruments does not include one or more medical instrument of the first type and the secondary medical instruments includes one or more medical instruments of the first type, e.g. to decide whether to reposition and release the first medical instrument to the secondary location. The secondary location may correspond to a secondary tray.

The method may comprise, in accordance with the primary medical instruments not including one or more medical instrument of the first type and the secondary medical instruments not including one or more medical instruments of the first type and the tertiary medical instruments including one or more medical instruments of the first type, repositioning the first medical instrument to a tertiary location for forming the tertiary set of medical instruments; and releasing the first medical instrument at the tertiary location. For example, the method may comprise determining whether the primary medical instruments does not include one or more medical instrument of the first type and the secondary medical instruments does not include one or more medical instruments of the first type and the tertiary medical instruments including one or more medical instruments of the first type, e.g. to decide whether to reposition and release the first medical instrument to the tertiary location. The tertiary location may correspond to a tertiary tray.

The method may comprise, in accordance with none of the medical instruments of the one or more sets of medical instruments including one or more medical instrument of the first type, repositioning the first medical instrument to a remainder location, e.g. for forming a remainder set of medical instruments, and releasing the first medical instrument at the remainder location. For example, the method may comprise determining whether none or the primary medical instruments includes one or more medical instruments of the first type, e.g. to decide whether to reposition and release the first medical instrument to the remainder location. The remainder location may correspond to a remainder tray. The remainder tray may be for collecting instruments not currently needed. After receiving further requests, the method may comprise identifying medical instruments from the remainder set of medical instruments, e.g. to be repositioned to form part of set of medical instruments according to the further requests.

Repositioning and/or releasing of medical instruments, such as the first medical instrument and/or a second medical instrument, may be provided by a reposition mechanism, e.g. a gripper or a suction device, such as the first reposition mechanism of the disclosed system.

The method may comprise identifying, e.g. with the electronic reader, such as the electronic reader of the disclosed system, a second medical instrument of a second type from the plurality of non-sorted medical instruments.

The method may comprise, in accordance with the primary medical instruments including one or more medical instruments of the second type, repositioning, e.g. by the reposition mechanism, such as the first reposition mechanism of the disclosed system, the second medical instrument to the primary location, and releasing the second medical instrument at the primary location. For example, the method may comprise determining whether the primary medical instruments include one or more medical instruments of the second type, e.g. to decide whether to reposition and release the second medical instrument to the primary location.

The method may comprise, in accordance with the primary medical instruments not including one or more medical instrument of the second type and the secondary medical instruments including one or more medical instruments of the second type, repositioning the second medical instrument to the secondary location, and releasing the second medical instrument at the secondary location. For example, the method may comprise determining whether the primary medical instruments does not include one or more medical instrument of the second type and the secondary medical instruments includes one or more medical instruments of the second type, e.g. to decide whether to reposition and release the second medical instrument to the secondary location.

The method may comprise, in accordance with the primary medical instruments not including one or more medical instrument of the second type and the secondary medical instruments not including one or more medical instruments of the second type and the tertiary medical instruments including one or more medical instruments of the second type, repositioning the second medical instrument to a tertiary location for forming the tertiary set of medical instruments; and releasing the second medical instrument at the tertiary location. The tertiary location may correspond to a tertiary tray. For example, the method may comprise determining whether the primary medical instruments does not include one or more medical instrument of the second type and the secondary medical instruments does not include one or more medical instruments of the second type and the tertiary medical instruments includes one or more medical instruments of the second type, e.g. to decide whether to reposition and release the second medical instrument to the tertiary location.

The method may comprise, in accordance with none of the medical instruments of the one or more sets of medical instruments including one or more medical instrument of the second type, repositioning the second medical instrument to the remainder location, and releasing the second medical instrument at the remainder location. For example, the method may comprise determining whether none of the medical instruments of the one or more sets of medical instruments includes one or more medical instrument of the second type, e.g. to decide whether to reposition and release the second medical instrument to the remainder location.

The system may comprise a first robotic arm. A retriever mechanism of the first reposition mechanism may be positioned at a first primary end of the first robotic arm. The retriever mechanism may be configured to retrieve the medical instrument, e.g. grip or attach to the medical instrument. The first robotic arm may form part of the first reposition mechanism.

The electronic reader may be arranged to form the identifier location of the electronic reader at the primary end of the first robotic arm. For example, the identifier location may be a location relative to the primary end of the first robotic arm. Alternatively, the identifier location may be relative to the initial location and/or the primary location, such as between the initial location and the primary location. The identifier location may be fixed relative to the initial location and/or the primary location, such as between the initial location and the primary location. For example, the identifier location may be provided between the initial location and the primary location.

Identifying with the electronic reader, such as the electronic reader of the disclosed system, may comprise repositioning the respective medical instrument to the identifier location of the electronic reader. For example, identifying the first medical instrument of the first type may comprise repositioning the first medical instrument to the identifier location, and/or identifying the second medical instrument of the second type may comprise repositioning the second medical instrument to the identifier location. The system may be configured to reposition, e.g. using the first reposition mechanism, the first and/or second medical instrument to the identifier location to identify with the electronic reader the type of the respective first and/or second medical instrument.

The system may comprise a second reposition mechanism. The second reposition mechanism, e.g. a gripper or suction device, may be configured to reposition a medical instrument comprising an electronic identification tag. The second reposition mechanism may comprise a second retriever mechanism. The system may comprise a second robotic arm, and the second retriever mechanism may be positioned at a second primary end of the second robotic arm. The second robotic arm may form part of the second reposition mechanism.

The method may comprise, e.g. prior to identifying the first medical instrument, arranging the plurality of non-sorted medical instruments to be individually spaced. Arranging the non-sorted medical instruments to be individually spaced may aid in correct identification of the medical instruments. Arranging the plurality of non-sorted medical instruments may comprise positioning each of the non-sorted medical instruments along a line, e.g. on a conveyor belt, such as a conveyor belt of the system. The second reposition mechanism may be configured to arrange the plurality of non-sorted medical instruments to be individually spaced, e.g. along the conveyor belt.

The identifier location may be provided relative to the conveyor belt such that medical instruments on the conveyor belt are identified by the electronic reader by passing the identifier location. For example, the identifier location may be provided below or above a part of the conveyor belt

### BRIEF DESCRIPTION OF THE FIGURES

Embodiments of the disclosure will be described in more detail in the following with regard to the accompanying figures. The figures show one way of implementing the present invention and are not to be construed as being limiting to other possible embodiments falling within the scope of the attached claim set.

FIG. 1 schematically illustrates an exemplary medical instrument,

FIG. 2 schematically illustrates a tray comprising medical instruments,

FIGS. 3a-c schematically illustrates an exemplary system,

FIGS. 4a-b schematically illustrates an exemplary system,

FIGS. 5a-b schematically illustrates an exemplary system

FIG. 6 schematically illustrated an exemplary reposition mechanism, and

FIG. 7 shows a block diagram of an exemplary method.

### DETAILED DESCRIPTION

Various exemplary embodiments and details are described hereinafter, with reference to the figures when relevant. It should be noted that the figures may or may not be drawn to scale and that elements of similar structures or functions are represented by like reference numerals throughout the figures. It should also be noted that the figures are only intended to facilitate the description of the embodiments. They are not intended as an exhaustive description of the invention or as a limitation on the scope of the invention. In addition, an illustrated embodiment needs not have all the aspects or advantages shown. An aspect or an advantage described in conjunction with a particular embodiment is not necessarily limited to that embodiment and can be practiced in any other embodiments even if not so illustrated, or if not so explicitly described.

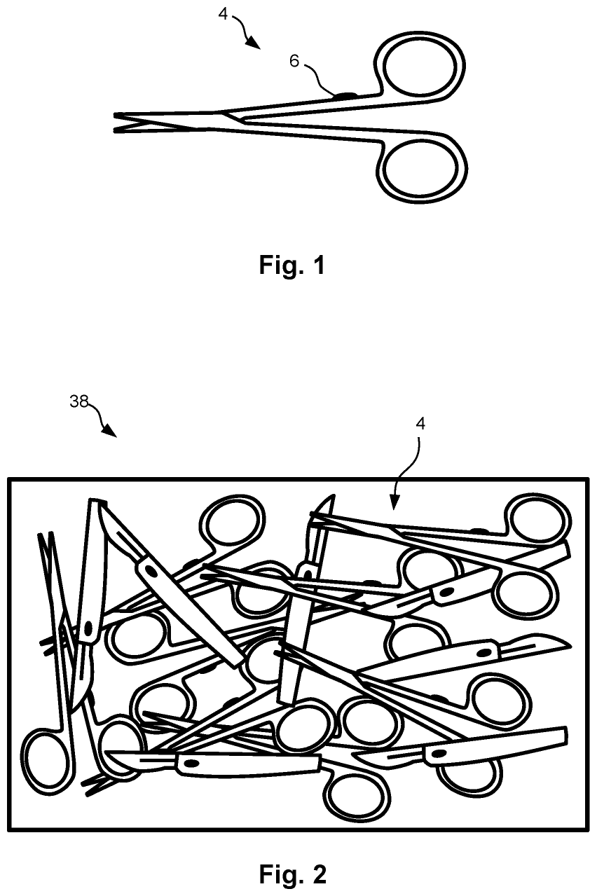

FIG. 1 schematically illustrates an exemplary medical instrument 4, e.g. a scissor in the illustrated example. The medical instrument 4 comprises an electronic identification tag 6, e.g. an RFID tag. The electronic identification tag 6 may be electronically readable, e.g. by electromagnetic radiation, and the information retrievable from the electronic identification tag 6 may indicate the type of the medical instrument 4, e.g. a scissor, a clamp, a scalpel, etc. The electronic identification tag may comprise different information, such as a unique identification number. A system may determine the type of the medical instrument 4 based on a unique identification number, and subsequently lookup the instrument based on the identification number to retrieve the type of the medical instrument. In another example, the type may be retrievable directly from the information stored in the electronic identification tag 6.

FIG. 2 schematically illustrates a tray 38 comprising a plurality of medical instruments 4, e.g. comprising different types, scalpels and scissors in the illustrated example. The tray 38 comprises medical instruments 4 not being sorted, i.e. chaotically positioned in the tray 38. Each of the non-sorted medical instruments 4 comprises an electronic identification tag 6, as described with respect to FIG. 1.

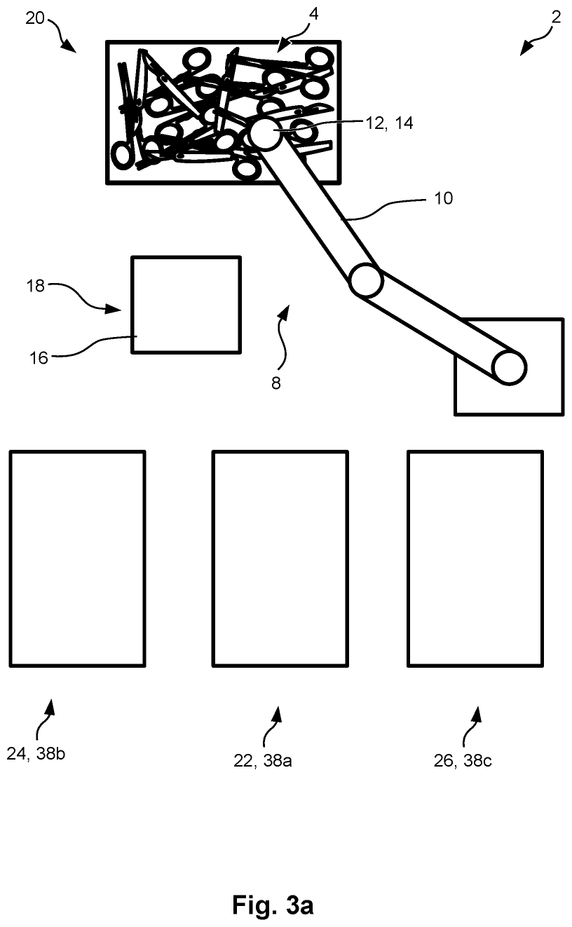

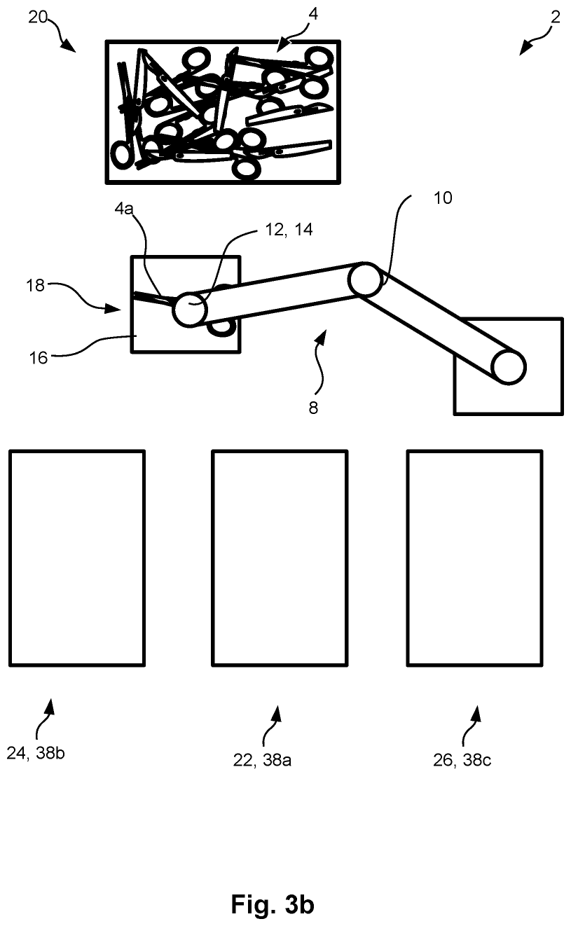

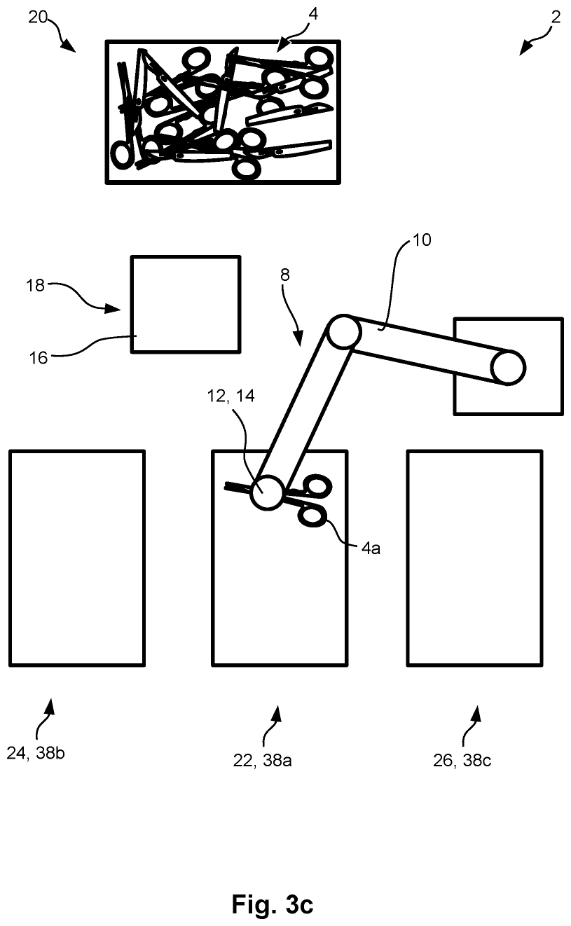

FIGS. 3a-c schematically illustrates an exemplary system 2, such as a system 2 for providing one or more sets of medical instruments 4. In the illustrated example, the system 2 is configured to provide a primary set, a secondary set, and a tertiary set. The primary set is to be provided in a primary tray 38a positioned at a primary location 22. The secondary set is to be provided in a secondary tray 38b positioned at a secondary location 24. The tertiary set is to be provided in a tertiary tray 38c positioned at a tertiary location 26.

The system 2 is configured to receive a plurality of non-sorted medical instruments 4 at an initial location 20.

The system 2 is configured to receive one or more requests indicative of one or more, such as a plurality of, medical instruments of the one or more sets of medical instruments, i.e. the system receives a request indicative of the medical instruments to be provided in each of the sets, e.g. the primary set, the secondary set, and/or the tertiary set. One of the sets, e.g. the tertiary set may alternatively function as a remainder set, i.e. a set which may be formed by medical instruments received but not being included in any of the requested sets. The requests may be received electronically, e.g. from a server. The system may comprise a processing unit (not shown) receiving the request and configured to control the elements of the system 2 accordingly.

The system 2 comprises first reposition mechanism 8 configured to reposition a medical instrument. The first reposition mechanism 8 may, as illustrated, comprise a first robotic arm 10. The first reposition mechanism 8 may further comprise a first retriever mechanism 12, e.g. a gripper or suction device, configured to retrieve, such as selectively hold, a medical instrument 4. The first retriever mechanism 12 is positioned at a first primary end 14 of the first robotic arm 10.

The system 2 comprises an electronic reader 16 configured to read the electronic identification tags 6. The electronic reader 16 forms an identifier location 18 wherein the electronic reader 16 may read the electronic identification tags 6 by emitting and receiving electromagnetic radiation. As exemplified in the system 2 as illustrated in FIGS. 3a-c, the electronic reader 16 and/or the identifier location 18 may be positioned relative to the initial location 20 and/or relative to a primary location 22 corresponding to the primary set, a secondary location 24 corresponding to the secondary set and/or a tertiary location 26 corresponding to the tertiary set. For example, the electronic reader 16 and/or the identifier location 18 may be positioned between the initial location 20 and one or more of the primary location 22, the secondary location 24 and the tertiary location 26.

The system 2 is configured to identify with the electronic reader 16 a first medical instrument 4a of a first type. As illustrated in FIG. 3a, the system 2 retrieves the first medical instrument 4a at the initial location 20 from the plurality of non-sorted medical instruments 4. As illustrated in FIG. 3b, the system 2 repositions, using the first reposition mechanism 8, the first medical instrument 4a to the identifier location 18 to identify with the electronic reader 16 the type of the first medical instrument 4a.

The system 2 hereafter determines which of the sets needs a medical instrument of the type of the first medical instrument 4a. In the present example, the primary medical instruments of the primary set includes a medical instruments of the first type, and the reposition mechanism 8, as illustrated in FIG. 3c, repositions the first medical instrument 4a to the primary tray 38a at the primary location 22 corresponding to the primary set of medical instruments, and releases the first medical instrument 4a at the primary location 22.

In another example, the primary medical instruments may have not included a medical instrument of the first type, and the system may have determined whether the secondary medical instruments of the secondary set included a medical instrument of the second type. Had also the secondary medical instruments not included a medical instrument of the first type, the system could have determined to use the tertiary location 26 corresponding to the tertiary tray 38c as a remainder location for forming a remainder set, repositioned the first medical instrument 4a to the tertiary location 26 and released the first medical instrument 4a at the tertiary location 26.

After having repositioned the first medical instrument 4a at an appropriate location, the system may continue by retrieving another, e.g. a second, medical instrument of non-sorted medical instrument and repeat the same procedure.

After having completed a set, the completed set may be removed by removing the tray. The completed tray may be replaced by a new empty tray to be packed according to another request.

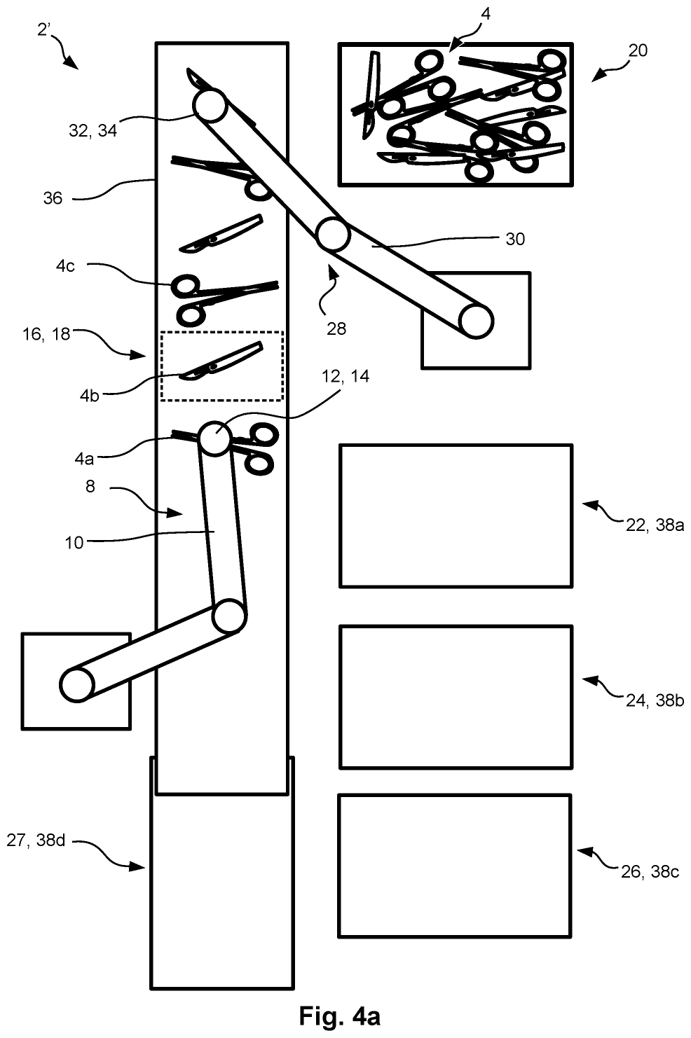

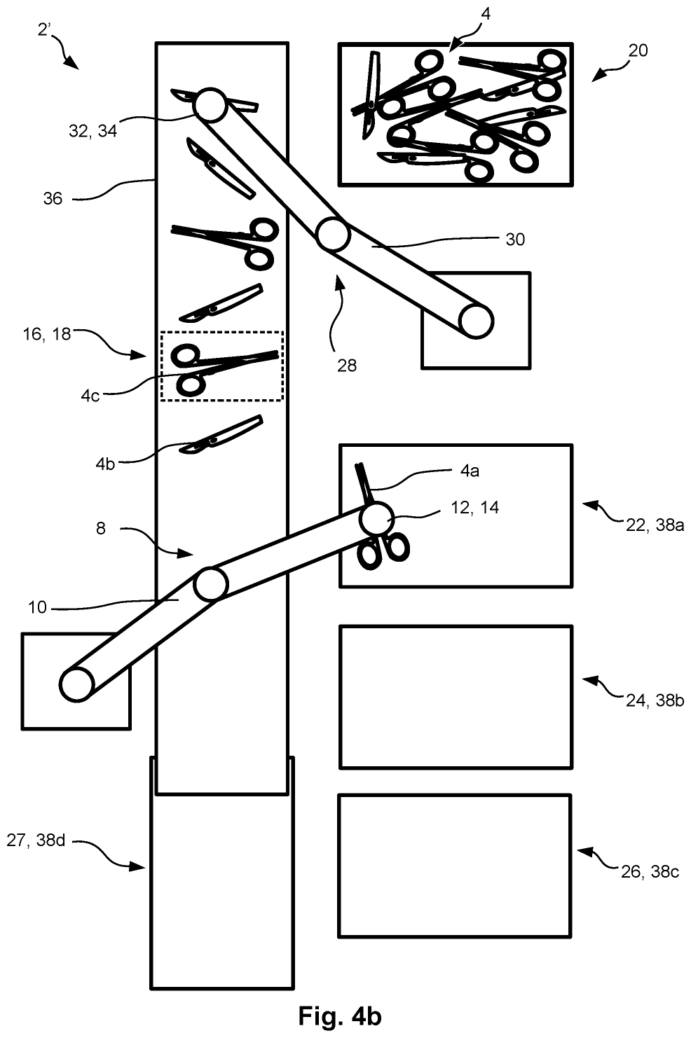

FIGS. 4a-b schematically illustrates another exemplary system 2′, such as a system 2′ for providing one or more sets of medical instruments 4. In the illustrated example, the system 2′ is configured to provide a primary set, a secondary set, and a tertiary set. The primary set is to be provided in a primary tray 38a positioned at a primary location 22. The secondary set is to be provided in a secondary tray 38b positioned at a secondary location 24. The tertiary set is to be provided in a tertiary tray 38c positioned at a tertiary location 26.

The system 2′ is configured to receive a plurality of non-sorted medical instruments 4 at an initial location 20.

The system 2′ is configured to receive one or more requests indicative of one or more, such as a plurality of, medical instruments of the one or more sets of medical instruments, i.e. the system receives a request indicative of the medical instruments to be provided in each of the sets, e.g. the primary set, the secondary set, and/or the tertiary set. The system 2′ also comprises a remainder tray 38d at a remainder location 27 wherein medical instruments of a type not forming part of any of the requested sets may be positioned to form a remainder set, e.g. to be used for later received requests.

The requests may be received electronically, e.g. from a server. The system 2′ may comprise a processing unit (not shown) receiving the request and configured to control the elements of the system 2′ accordingly.

The system 2′ comprises a first reposition mechanism 8 configured to reposition a medical instrument 4. The first reposition mechanism 8 may, as illustrated, comprise a first robotic arm 10. The first reposition mechanism 8 may further comprise a first retriever mechanism 12, e.g. a gripper or suction device, configured to retrieve, such as selectively hold, a medical instrument 4. The first retriever mechanism 12 is positioned at a first primary end 14 of the first robotic arm 10.

The system 2′ comprises an electronic reader 16 configured to read the electronic identification tags 6. The electronic reader 16 forms an identifier location 18 wherein the electronic reader 16 may read the electronic identification tags 6 by emitting and receiving electromagnetic radiation. As exemplified in the system 2′ as illustrated in FIGS. 4a-b, the electronic reader 16 and/or the identifier location 18 may be positioned relative to the initial location 20 and/or relative to the primary location 22 corresponding to the primary set, the secondary location 24 corresponding to the secondary set, the tertiary location 26 corresponding to the tertiary set and/or the remainder location 27 corresponding to the remainder set. For example, the electronic reader 16 and/or the identifier location 18 may be positioned between the initial location 20 and one or more of the primary locations 22, the secondary location 24, the tertiary location 26 and the remainder location 27. In the illustrated example, the system 2′ comprises a conveyor belt 36 on which the medical instruments 4 are arranged, and the identifier location 18 is provided relative to the conveyor belt 36, e.g. the electronic reader 16 may be provided below the conveyor belt, such that the medical instruments 4 on the conveyor belt 36 are read by the electronic reader 16 when passing the identifier location 18.

The system 2′ comprises a second reposition mechanism 28 configured to reposition a medical instrument 4. Particularly, the second reposition mechanism is configured to arrange the plurality of non-sorted medical instruments 4 to be individually spaced. For example, by arranging the non-sorted medical instruments 4 to be individually spaced along a line on the conveyor belt 36. The second reposition mechanism 28 may, as illustrated, comprise a second robotic arm 30. The second reposition mechanism 28 may further comprise a second retriever mechanism 32, e.g. a gripper or suction device, configured to retrieve, such as selectively hold, a medical instrument 4. The second retriever mechanism 32 is positioned at a second primary end 34 of the second robotic arm 30.

As illustrated in FIG. 4a, the second reposition mechanism 28 has arranged a plurality of medical instruments, including a first medical instrument 4a, a second medical instrument 4b, and a third medical instrument 4c on the conveyor belt 36, such that the medical instruments are individually spaced along the conveyor belt 36. The system 2′ is configured to identify with the electronic reader 16 each of the medical instruments, in FIG. 4a, the system 2′ is identifying the second medical instrument 4b, and previously the system 2′ has identified the first medical instrument 4a when it passed by the identifier location 18.

The system 2′ determines which of the sets needs a medical instrument of the type of the first medical instrument 4a. In the present example, the primary medical instruments of the primary set includes a medical instruments of the first type, and the reposition mechanism 8, as illustrated in

FIG. 4b, repositions the first medical instrument 4a to the primary tray 38a at the primary location 22 corresponding to the primary set of medical instruments, and releases the first medical instrument 4a at the primary location 22.

In another example, the primary medical instruments may have not included a medical instrument of the first type, and the system 2′ may have determined whether the secondary medical instruments of the secondary set included a medical instrument of the second type. Had also the secondary medical instruments not included a medical instrument of the first type, the system 2′ could have determined to use the tertiary location 26 corresponding to the tertiary tray 38c as a remainder location for forming a remainder set, repositioned the first medical instrument 4a to the tertiary location 26 and released the first medical instrument 4a at the tertiary location 26.

After having repositioned the first medical instrument 4a at an appropriate location, the system 2′ may continue by determining which of the sets needs a medical instrument of the type of the second medical instrument 4b, then the third medical instrument 4c, etc.

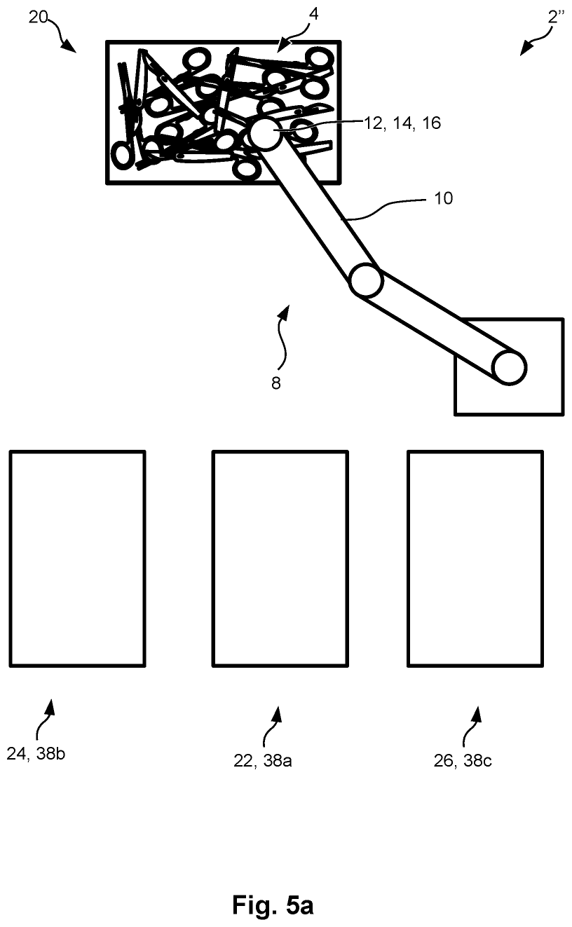

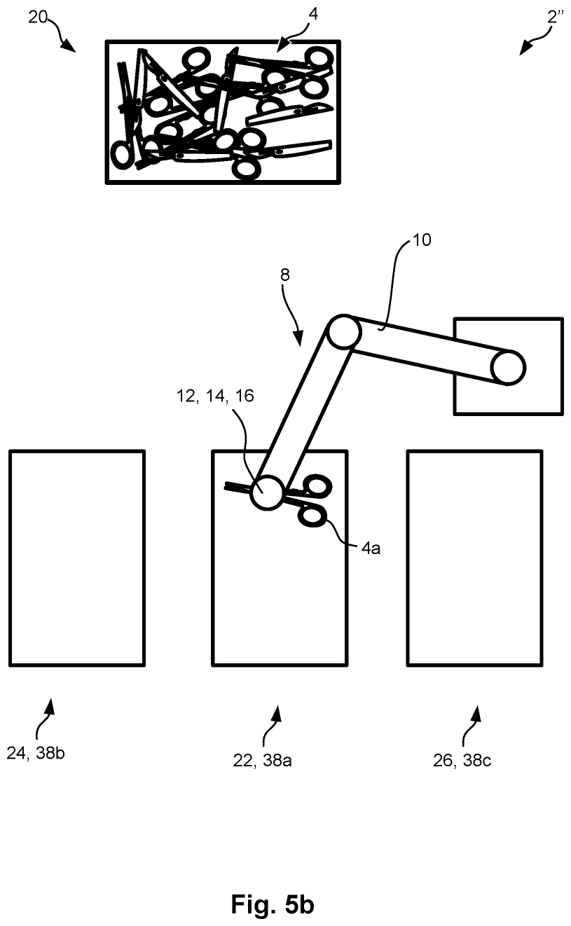

FIGS. 5a-b schematically illustrates another exemplary system 2″, such as a system 2″ for providing one or more sets of medical instruments 4. The system 2″ resembles the system 2 as illustrated in FIGS. 3a-c. However, with the only difference that the electronic reader 16 is arranged to form the identifier location 18 of the electronic reader 16 at the first primary end 14 of the first robotic arm 10. Thereby the electronic reader and/or the identifier location is movable with the first primary end 14 of the first robotic arm and the first retriever mechanism. Thereby, identification of the medical instrument may be done prior to retrieving it, and a medical instrument may be selectively retrieved from the non-sorted medical instruments 4 based on the type of medical instrument needed for a requested set. Consequently, the system 2″ of FIGS. 5a-b may not need to reposition the first medical instrument 4a to an identifier location 18 as in FIGS. 3a-c for identification.

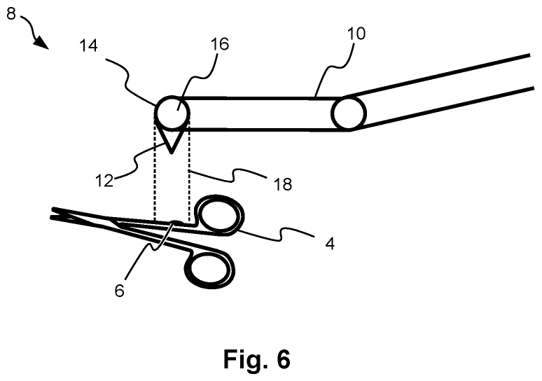

FIG. 6 schematically illustrated an exemplary reposition mechanism 8 in accordance with the system 2″ as illustrated in FIG. 5a-b, wherein the electronic reader 16 is arranged to form the identifier location 18 of the electronic reader 16 at the first primary end 14 of the first robotic arm 10. To distinguish between medical instruments located close together, the electronic reader 16 is configured to read electronic identification tags 6 located at a very small identifier location.

With the electronic reader 16 at the primary end of the first robotic arm and/or at a location relative to the primary end of the first robot arm, such as to form the identifier location 18 at the first primary end 14, a specific instrument may be located prior to picking up the instrument. For example, this may be done by searching for the specific instrument by moving the primary end 14 of the first robotic arm 10 over the plurality of non-sorted medical instruments, while continuously emitting and receiving electromagnetic radiation. In the position where the response is strongest and/or above a set threshold, the instrument underneath that position may be picked up. Confirmation of the desired instrument being picked up may be performed by again emitting and receiving electromagnetic radiation once the instrument is repositioned away from the remainder of the non-sorted medical instruments.

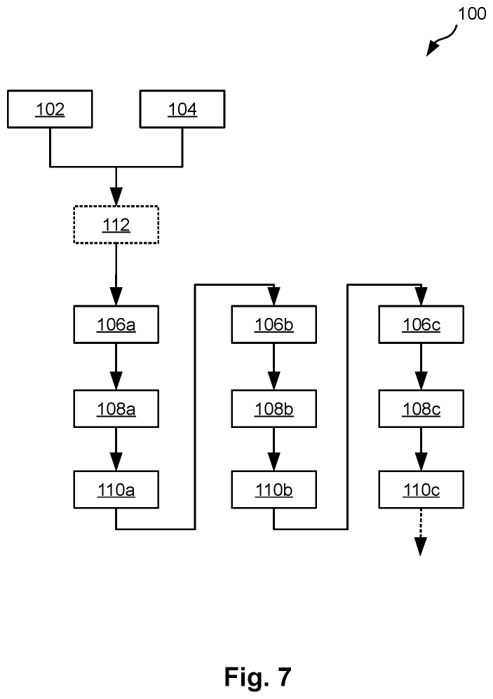

FIG. 7 shows a block diagram of an exemplary method 100, such as a method 100 for providing one or more sets of medical instruments. The method may be performed by the systems 2, 2′, 2″ as exemplified with respect to FIGS. 3-5.

The method 100 comprises receiving 102 one or more requests indicative of one or more, such as a plurality of, medical instruments of one or more sets of medical instruments. The one or more received request 102 may comprise a primary request indicative of one or more, such as a plurality of, primary medical instruments to be included in a primary set of medical instruments. The one or more received request 102 may comprise a secondary request indicative of one or more, such as a plurality of, secondary medical instruments to be included in a secondary set of medical instruments. The one or more received request 102 may comprise a tertiary request indicative of one or more, such as a plurality of, tertiary medical instruments to be included in a tertiary set of medical instruments.

The method 100 comprises receiving 104 a plurality of non-sorted medical instruments at an initial location. The non-sorted medical instruments comprise electronic identification tags. Receiving 104 the non-sorted medical instruments may be independent of receiving 102 the one or more requests, i.e. receiving 104 the non-sorted medical instruments may be before, after or simultaneously with receiving 102 the one or more requests.

The method 100 comprises identifying 106a with an electronic reader, such as the electronic reader of the above described systems, a first medical instrument of a first type from the received 104 plurality of non-sorted medical instruments.

The method 100 comprises repositioning 108a the first medical instrument, by a reposition mechanism, such as the first and/or second reposition mechanism of the above described systems, to a primary location for forming the primary set of medical instruments, to a secondary location for forming the secondary set of medical instruments, to a tertiary location for forming the tertiary set of medical instruments or to a remainder location, in accordance with requests.

In accordance with the primary medical instruments including one or more medical instruments of the first type, the first medical instrument may be repositioned 108a to the primary location for forming the primary set of medical instruments.

In accordance with the primary medical instruments not including one or more medical instrument of the first type and the secondary medical instruments including one or more medical instruments of the first type, the first medical instrument may be repositioned 108a to the secondary location for forming the secondary set of medical instruments.

In accordance with the primary medical instruments not including one or more medical instrument of the first type and the secondary medical instruments not including one or more medical instruments of the first type and the tertiary medical instruments including one or more medical instruments of the first type, the first medical instrument may be repositioned 108a to the tertiary location for forming the tertiary set of medical instruments.

In accordance with none of the medical instruments of the one or more sets of medical instruments including one or more medical instrument of the first type, the first medical instrument may be repositioned 108a to the remainder location.

The method 100 comprises releasing 110a the first medical instrument at the respective primary, secondary, tertiary or remainder location.

The method 100 may continue with identifying 106b a second medical instrument, repositioning 108b the second medical instrument, and releasing 110b the second medical instrument. Next the method 100 may continue with identifying 106c a third medical instrument, repositioning 108c the third medical instrument, and releasing 110c the third medical instrument, and so forth.

The method 100 may comprise arranging 112 the plurality of non-sorted medical instruments to be individually spaced. For example, such as exemplified in FIGS. 4a-b, wherein the non-sorted medical instruments are arranged by positioning each of the non-sorted medical instruments along a line on a conveyor belt.

The invention has been described with reference to a preferred embodiment. However, the scope of the invention is not limited to the illustrated embodiment, and alterations and modifications can be carried out without deviating from the scope of the invention.

Throughout the description, the use of the terms “first”, “second”, “third”, “fourth”, “primary”, “secondary”, “tertiary” etc. does not imply any particular order or importance but are included to identify individual elements. Furthermore, the labelling of a first element does not imply the presence of a second element and vice versa.

### LIST OF REFERENCES

2, 2′, 2″ system

4, 4a, 4b, 4c medical instrument

6 electronic identification tag

8 first reposition mechanism

10 first robotic arm

12 first retriever mechanism

14 first primary end

16 electronic reader

18 identifier location

20 initial location

22 primary location

24 secondary location

26 tertiary location

27 remainder location

28 second reposition mechanism

30 second robotic arm

32 second retriever mechanism

34 second primary end

36 conveyor belt

38, 38a, 38b, 38c, 38d tray

100 method

102 receiving request(s)

104 receiving non-sorted medical instruments

106a, 106b, 106c identifying medical instrument

108a, 108b, 108c repositioning medical instrument

110a, 110b, 110c releasing medical instrument

112 arranging non-sorted medical instruments

1. A method for providing one or more sets of medical instruments including a primary set of medical instruments, the method comprising:
receiving one or more requests indicative of one or more medical instruments of the one or more sets of medical instruments, the one or more requests including a primary request indicative of one or more primary medical instruments to be included in the primary set of medical instruments;
receiving a plurality of non-sorted medical instruments at an initial location, wherein each of the non-sorted medical instruments comprises an electronic identification tag, e.g. an RFID tag;
identifying with an electronic reader configured for reading electronic identification tags by emitting and receiving electromagnetic radiation within a first frequency range, a first medical instrument of a first type from the plurality of non-sorted medical instruments; and
in accordance with the primary medical instruments including one or more medical instruments of the first type:
repositioning, by a reposition mechanism, e.g. a gripper or suction device, the first medical instrument to a primary location for forming the primary set of medical instruments; and
releasing the first medical instrument at the primary location.

2. Method according to claim 1, wherein the one or more requests includes a secondary request indicative of one or more secondary medical instruments to be included in a secondary set of medical instruments of the one or more sets of medical instruments.

3. Method according to claim 2 comprising in accordance with the primary medical instruments not including one or more medical instrument of the first type and the secondary medical instruments including one or more medical instruments of the first type:
repositioning, by the reposition mechanism, the first medical instrument to a secondary location for forming the secondary set of medical instruments; and
releasing the first medical instrument at the secondary location.

4. Method according to claim 1 comprising in accordance with none of the medical instruments of the one or more sets of medical instruments including one or more medical instrument of the first type:
repositioning, by the reposition mechanism, the first medical instrument to a remainder location; and
releasing the first medical instrument at the remainder location.

5. Method according to claim 1 comprising identifying with the electronic reader a second medical instrument of a second type from the plurality of non-sorted medical instruments, and in accordance with the primary medical instruments including one or more medical instruments of the second type:
repositioning, by the reposition mechanism the second medical instrument to the primary location; and
releasing the second medical instrument at the primary location.

6. Method according to claim 1 comprising, prior to identifying the first medical instrument, arranging the plurality of non-sorted medical instruments to be individually spaced.

7. Method according to claim 6, wherein arranging the plurality of non-sorted medical instruments comprises positioning each of the non-sorted medical instruments along a line, e.g. on a conveyor belt.

8. Method according to claim 1 wherein identifying with the electronic reader comprises repositioning the respective medical instrument to an identifier location of the electronic reader.

9. Method according to claim 8, wherein the identifier location is fixed relative to the initial location and/or the primary location, such as between the initial location and the primary location.

10. System for providing one or more sets of medical instruments including a primary set of medical instruments, the system comprising:
a first reposition mechanism, e.g. a gripper or suction device, configured to reposition a medical instrument comprising an electronic identification tag, e.g. an RFID tag
an electronic reader configured to read electronic identification tags at an identifier location by emitting and receiving electromagnetic radiation within a first frequency range, wherein the system is configured to perform the method of any of claims 1-9.

11. System according to claim 10 comprising a first robotic arm, and wherein a retriever mechanism of the first reposition mechanism is positioned at a first primary end of the first robotic arm, the retriever mechanism is configured to retrieve the medical instrument.

12. System according to claim 11, wherein the electronic reader is arranged to form the identifier location of the electronic reader at the primary end of the first robotic arm.

13. System according to claim 10, wherein the identifier location is fixed relative to the initial location and/or the primary location, such as between the initial location and the primary location.

14. System according to claim 13, wherein the system is configured to reposition, using the first reposition mechanism, the first medical instrument to the identifier location to identify with the electronic reader the type of the first medical instrument.

15. System according to claim 10 comprising a second robotic arm, and wherein a second reposition mechanism comprises a second retriever mechanism positioned at a second primary end of the second robotic arm.

16. System according to claim 15 comprising a conveyor belt, and wherein the second reposition mechanism is configured to arrange the plurality of non-sorted medical instruments to be individually spaced along the conveyor belt.

17. System according to claim 16, wherein the identifier location is provided relative to the conveyor belt such that medical instruments on the conveyor belt are identified by the electronic reader by passing the identifier location.
